(19) United States
(12) Patent Application Publication
(10) Pub. No.: US 2006/0167382 A1
(43) Pub. Date: Jul. 27, 2006

(54) Method and apparatus for storing an analyte sampling and measurement device

(76) Inventor: Ajay Deshmukh, San Francisco, CA (US)

Correspondence Address: HELLER EHRMAN LLP, 275 MIDDLEFIELD ROAD, MENLO PARK, CA, 94025-3506, US

(21) Appl. No.: 11/324,001

(22) Filed: Dec. 29, 2005

Related U.S. Application Data
(60) Provisional application No. 60/640,839, filed on Dec. 30, 2004.

(51) Int. Cl. A61B 5/00

(52) U.S. Cl. 600/583; 600/584

(57) ABSTRACT

Methods and apparatus are provided for storing an analyte sampling and measurement device. In one embodiment, an analyte sampling device has a housing and a cartridge having a plurality of penetrating members wherein the penetrating members are slidably movable to extend outward from lateral openings on said cartridge to penetrate tissue, where the sampling device include a plurality of analyte detecting members. The device is fitted with a plurality of gaskets to provide a sealed environment inside the sampling device when the device is not in use. The user can open a lid to allow for lancing and sample capture. The lid is closed to re-establish a sealed condition inside the device once lancing is complete.

### CROSS-REFERENCE TO RELATED APPLICATIONS

This application claims the benefit of U.S. Ser. No. 60/640,839, filed Dec. 30, 2004, which application is fully incorporated herein by reference.

### BACKGROUND OF THE INVENTION

1. Technical Field

The technical field relates to analyte sampling devices, and more specifically, methods and devices for storing analyte sampling and measurement devices in a safe, usable condition.

2. Background Art

Lancing devices are known in the medical health-care products industry for piercing the skin to produce blood for analysis. Typically, a drop of blood for this type of analysis is obtained by making a small incision in the fingertip, creating a small wound, which generates a small blood droplet on the surface of the skin.

Early methods of lancing included piercing or slicing the skin with a needle or razor. Current methods utilize lancing devices that contain a multitude of spring, cam and mass actuators to drive the lancet. These include cantilever springs, diaphragms, coil springs, as well as gravity plumbs used to drive the lancet. The device may be held against the skin and mechanically triggered to ballistically launch the lancet. Unfortunately, the pain associated with each lancing event using known technology discourages patients from testing. In addition to vibratory stimulation of the skin as the driver impacts the end of a launcher stop, known spring based devices have the possibility of firing lancets that harmonically oscillate against the patient tissue, causing multiple strikes due to recoil. This recoil and multiple strikes of the lancet is one major impediment to patient compliance with a structured glucose monitoring regime.

Success rate generally encompasses the probability of producing a blood sample with one lancing action, which is sufficient in volume to perform the desired analytical test. The blood may appear spontaneously at the surface of the skin, or may be “milked” from the wound. Milking generally involves pressing the side of the digit, or in proximity of the wound to express the blood to the surface. In traditional methods, the blood droplet produced by the lancing action must reach the surface of the skin to be viable for testing.

When using existing methods, blood often flows from the cut blood vessels but is then trapped below the surface of the skin, forming a hematoma. In other instances, a wound is created, but no blood flows from the wound. In either case, the lancing process cannot be combined with the sample acquisition and testing step. Spontaneous blood droplet generation with current mechanical launching system varies between launcher types but on average it is about 50% of lancet strikes, which would be spontaneous. Otherwise milking is required to yield blood. Mechanical launchers are unlikely to provide the means for integrated sample acquisition and testing if one out of every two strikes does not yield a spontaneous blood sample.

Many diabetic patients (insulin dependent) are required to self-test for blood glucose levels five to six times daily. The large number of steps required in traditional methods of glucose testing ranging from lancing, to milking of blood, applying blood to the test strip, and getting the measurements from the test strip discourages many diabetic patients from testing their blood glucose levels as often as recommended. Tight control of plasma glucose through frequent testing is therefore mandatory for disease management. The pain associated with each lancing event further discourages patients from testing. Additionally, the wound channel left on the patient by known systems may also be of a size that discourages those who are active with their hands or who are worried about healing of those wound channels from testing their glucose levels.

Another problem frequently encountered by patients who must use lancing equipment to obtain and analyze blood samples is the amount of manual dexterity and hand-eye coordination required to properly operate the lancing and sample testing equipment due to retinopathies and neuropathies particularly, severe in elderly diabetic patients. For those patients, operating existing lancet and sample testing equipment can be a challenge. Once a blood droplet is created, that droplet must then be guided into a receiving channel of a small test strip or the like. If the sample placement on the strip is unsuccessful, repetition of the entire procedure including re-lancing the skin to obtain a new blood droplet is necessary.

Early methods of using test strips required a relatively substantial volume of blood to obtain an accurate glucose measurement. This large blood requirement made the monitoring experience a painful one for the user since the user may need to lance deeper than comfortable to obtain sufficient blood generation. Alternatively, if insufficient blood is spontaneously generated, the user may need to “milk” the wound to squeeze enough blood to the skin surface. Neither method is desirable as they take additional user effort and may be painful. The discomfort and inconvenience associated with such lancing events may deter a user from testing their blood glucose levels in a rigorous manner sufficient to control their diabetes.

A further impediment to patient compliance is the technique for storing these analyte sampling and analyte detecting devices. The devices used to measure analyte levels are typically stored in a humidity controlled or other safe environment to maintain the device shelf life. This often involves using a variety of containers, some for the test strips and some for the lancets. The introduction of multiple storage devices and the cumbersome design may discourage users from keeping their equipment in a usable condition, further degrading user test compliance and measurement accuracy.

There is a need for a device to measure analyte levels with improved humidity control. There is a further need for a device to measure analyte levels that includes desiccant that is external to penetrating members.

### SUMMARY OF THE INVENTION

Accordingly, an object of the present invention is to provide an improved fluid sampling device.

Another object of the present invention is to provide a fluid sampling device, and its methods of use, that provides a desiccated case for the entire instrument housing.

Yet another object of the present invention is to provide a fluid sampling device, and its methods of use, that includes a plurality of analyte detection members, a plurality of penetrating members, and a desiccant that is external to the plurality of penetrating members.

A further object of the present invention is to provide a fluid sampling device, and its methods of use, that includes a plurality of analyte detection members, a plurality of penetrating members, a desiccant that is external to the plurality of penetrating members and holds the desiccant.

These and other objects of the present invention are achieved in a fluid sampling device with an instrument housing. A plurality of penetrating members are in the instrument housing. A plurality of analyte detecting members are also included. Each of an analyte detecting member is coupled to a penetrating member. A desiccant material is inside the instrument housing and positioned external to the plurality of penetrating members.

In another embodiment of the present invention, a fluid sampling device has an instrument housing. A plurality of penetrating members are in the instrument housing. A plurality of analyte detecting members are also included. Each of an analyte detecting member is coupled to a penetrating member. A case is sized to contain the instrument housing. A desiccant material is inside the instrument housing or the case. The desiccant material is positioned external to the plurality of penetrating members.

In another embodiment of the present invention, a method determines an amount on an analyte in a body fluid sample by a user. An analyte measuring device is provided that has, a instrument housing, a plurality of penetrating members in the instrument housing, a plurality of analyte detecting members, a sterility barrier configured to provide sterile environments for the penetrating members and a desiccant material inside the instrument housing and positioned external to the plurality of penetrating members. The plurality of analyte detecting members are desiccated with the desiccant that is external to the plurality of penetrating members. A penetrating member and unused analyte detecting member of the analyte measurement device are presented into an active position. The penetrating member is fired to prick the skin and bring a fluid sample to the analyte detecting member. The analyte level is measured.

### BRIEF DESCRIPTION OF THE DRAWINGS

FIG. 1 is a perspective view illustrating one embodiment of a fluid sampling device with an instrument housing of the present invention.

FIG. 2 is a partial sectional view of a disposable device that can be utilized with the FIG. 1 device.

FIG. 3 is a full sectional view of the FIG. 2 disposable device.

FIG. 4 is an exploded view of a cartridge that can be utilized with the FIG. 1 device.

FIG. 5 illustrates the FIG. 1 device and a case.

FIG. 6 illustrates an embodiment of a penetrating member driver that can used with the FIG. 1 device.

FIGS. 7(a) and 7(b) illustrate embodiments of displacement and velocity profiles, respectively, of a harmonic spring/mass powered driver that can be used with the FIG. 1 device.

FIG. 7(c) illustrates an embodiment of a controlled displacement profile.

FIG. 7(d) illustrates an embodiment of a controlled velocity profile to be utilized with the present invention.

FIG. 8 illustrates a feedback loop and a processor that can be used with the FIG. 1 device.

FIG. 9 illustrates a tissue penetration device, more specifically, a lancing device and a controllable driver coupled to a tissue penetration element, that can be used with the FIG. 1 device.

FIG. 10 illustrates the lancing device of FIG. 9 in more detail.

### DESCRIPTION OF THE SPECIFIC EMBODIMENTS

The present invention provides a solution for body fluid sampling. Specifically, some embodiments of the present invention provide improved devices and methods for storing a sampling device. The invention may use a high density penetrating member design. It may use penetrating members of smaller size, such as but not limited to diameter or length, than those of conventional penetrating members known in the art. The device may be used for multiple lancing events without having to remove a disposable from the device. The invention may provide improved sensing capabilities. At least some of these and other objectives described herein will be met by embodiments of the present invention.

It is to be understood that both the foregoing general description and the following detailed description are exemplary and explanatory only and are not restrictive of the invention, as claimed. It may be noted that, as used in the specification and the appended claims, the singular forms “a”, “an” and “the” include plural referents unless the context clearly dictates otherwise. Thus, for example, reference to “a material” may include mixtures of materials, reference to “a chamber” may include multiple chambers, and the like. References cited herein are hereby incorporated by reference in their entirety, except to the extent that they conflict with teachings explicitly set forth in this specification.

In this specification and in the claims which follow, reference will be made to a number of terms which shall be defined to have the following meanings:

“Optional” or “optionally” means that the subsequently described circumstance may or may not occur, so that the description includes instances where the circumstance occurs and instances where it does not. For example, if a device optionally contains a feature for analyzing a blood sample, this means that the analysis feature may or may not be present, and, thus, the description includes structures wherein a device possesses the analysis feature and structures wherein the analysis feature is not present.

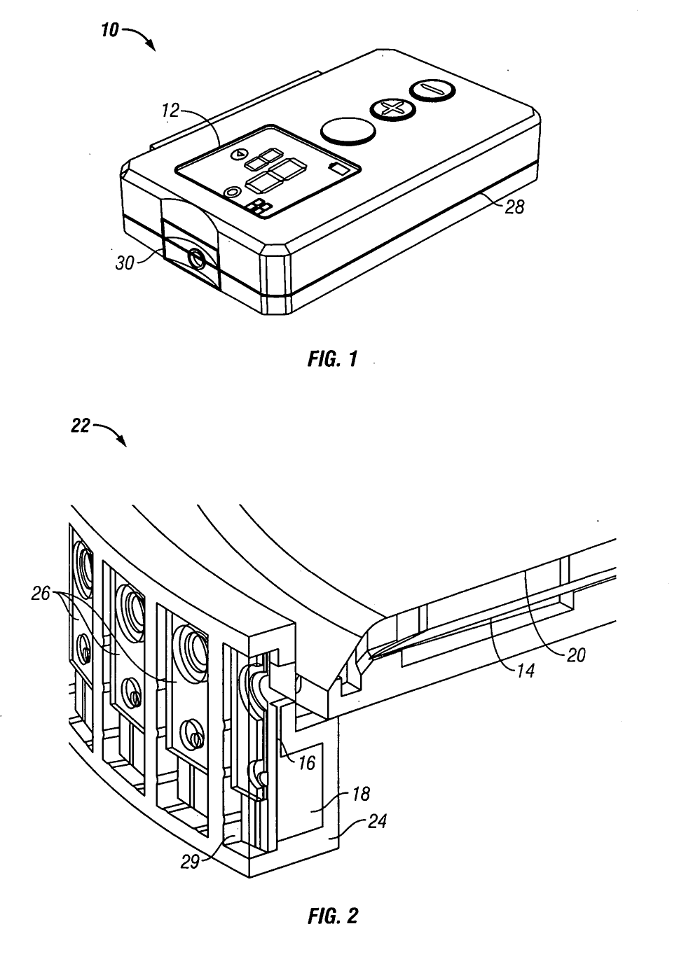

Referring to FIG. 1, one embodiment of the present invention is a fluid sampling device 10 with an instrument housing 12.

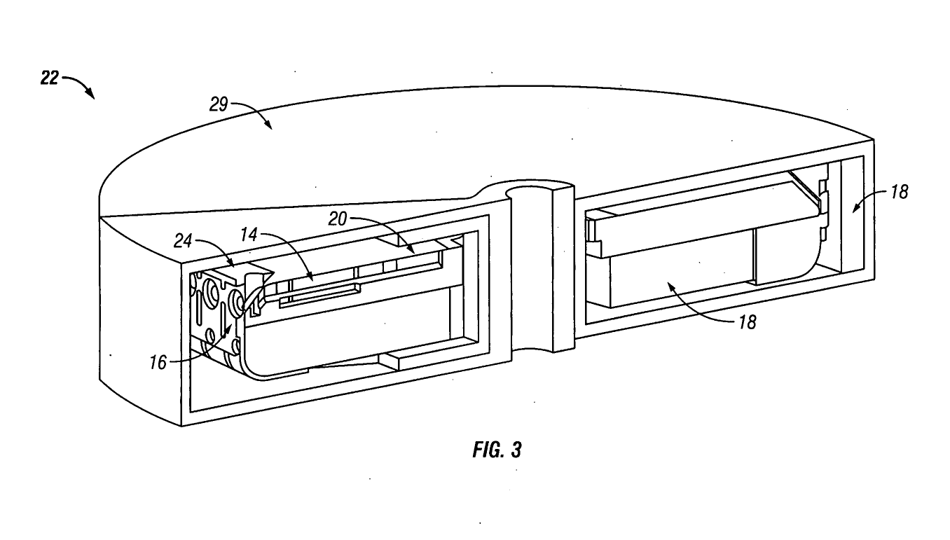

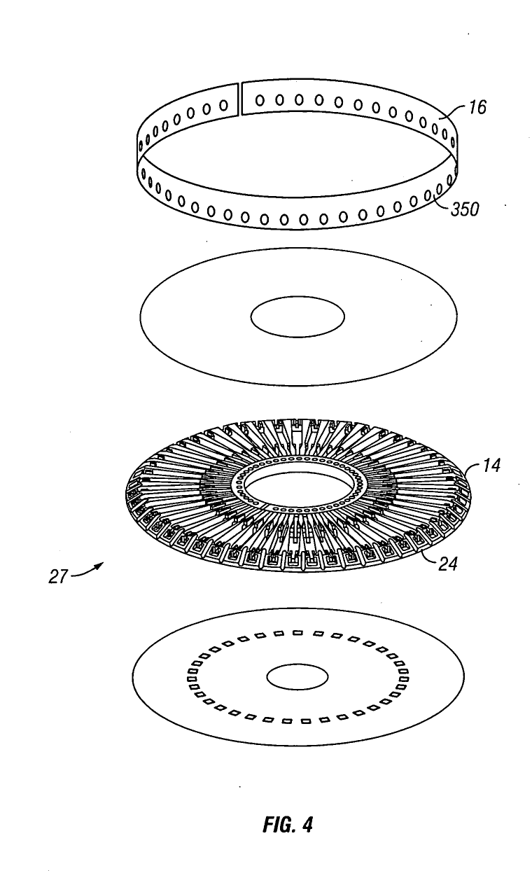

As shown in FIGS. 2 and 3, a plurality of penetrating members 14 are in the instrument housing 12. A plurality of analyte detecting members 16 are also included. Each of an analyte detecting member 16 is coupled to a penetrating member 14. A desiccant material 18 is inside the instrument housing 12 and positioned external to the plurality of penetrating members 14. A sterility barrier 20 is configured to provide sterile environments for the plurality of penetrating members 14. The sterility barrier 20 can be made of a variety of materials including but not limited to, a metallic foil or other seal materials and may be of a tensile strength and other quality that may provide a sealed, sterile environment until the sterility barrier 20 is penetrated by a penetrating device 14, providing a preselected or selected amount of force to open the sealed, sterile environment.

The plurality of analyte detecting members 16 and the plurality of penetrating members 14 can form a disposable device 22. The sterility barrier 20 can be a planar material that is adhered to a surface of the disposable device 22. Depending on the orientation of the disposable device 22, the sterility barrier 20 can be on the top surface, side surface, bottom surface, or other positioned surface of the disposable device 20. The desiccant material 18 can be configured to be replaced when the disposable device 22 is replaced from the instrument housing 12.

In various embodiments, the desiccant 18 is present in an amount of no more than, 50 mm³, 10-20 mm^{3,} 10-15 mm^{3,} at least 1 mm³ per each of an analyte detecting member 16 and the like. The desiccant 18 can be a variety of materials, including but not limited to, a molecular sieve, a silica gel, a clay, and the like. The molecular sieve can be mixed with a polymeric binder.

The plurality of analyte detecting members 16 can be supported on a scaffolding 24 (FIGS. 2 and 3). The scaffolding 24 can be attached to a bottom surface of the disposable device 22. The scaffolding 24 can be made of a material such as, but not limited to, a polymer, a foil, and the like. The scaffolding 24 can hold a plurality of analyte detecting members 16, such as but not limited to, about 10-50, 50-100, or other combinations of analyte detecting members 16. This facilitates the assembly and integration of analyte detecting members 16 with disposable device 22. These analyte detecting members 16 can enable an integrated body fluid sampling system where the penetrating members 14 create a wound tract in a target tissue, which expresses body fluid that flows into the disposable device 22 for analyte detection by at least one of the analyte detecting members 16.

In one embodiment, many analyte detecting members 16 can be printed onto a single scaffolding 22 which is then adhered to the disposable device 22 to facilitate manufacturing and simplify assembly. The analyte detecting members 16 can be electrochemical in nature. The analyte detecting members 16 can further contain enzymes, dyes, or other detectors which react when exposed to the desired analyte. Additionally, the analyte detecting members 16 can comprise of clear optical windows that allow light to pass into the body fluid for analyte analysis. The number, location, and type of analyte detecting member 16 can be varied as desired, based in part on the design of the disposable device 22, number of analytes to be measured, the need for analyte detecting member calibration, and the sensitivity of the analyte detecting members 16. Wicking elements, capillary tube or other devices on the disposable device 22 can be provided to allow body fluid to flow from the disposable device 22 to the analyte detecting members 16 for analysis. In other configurations, the analyte detecting members 16 can be printed, formed, or otherwise located directly in the disposable device 22.

In one embodiment, the desiccant material 18 is external to the analyte detecting members 16. The desiccant 18 can be on at least a portion of the analyte detecting members 16. In one embodiment, the scaffolding 24 holds the desiccant 18. In another embodiment, the scaffolding 24 includes a desiccant 18 for each of an analyte detecting member 16. Each of analyte detecting member 16 can be stored in an air tight desiccated environment.

The desiccant 18 can be molded and inserted into the scaffolding 24. In one embodiment, the desiccant 18 and the scaffolding 24 are co-molded simultaneously. In another embodiment, the scaffolding 24 and the desiccant 18 are co-molded sequentially. The desiccant 18 can be present as a desiccant block inside of the instrument housing 12.

As shown in FIGS. 2 and 3, the disposable device 22 can include a plurality of cavities 26. Each penetrating member 14 may be contained in a cavity 26 in the disposable device 22 with its sharpened end facing radially outward and may be in the same plane as that of the disposable device 22. The cavity 26 may be molded, pressed, forged, or otherwise formed in the disposable device 22. Although not limited in this manner, the ends of the cavities 26 may be divided into individual fingers (such as one for each cavity) on the outer periphery of the disposable device 22. The particular shape of each cavity 26 may be designed to suit the size or shape of the penetrating member therein or the amount of space desired for placement of the analyte detecting members 16. For example and not limitation, the cavity 26 may have a V-shaped cross-section, a U-shaped cross-section, C-shaped cross-section, a multi-level cross section or the other cross-sections. The opening through which a penetrating member 14 may exit to penetrate tissue may also have a variety of shapes, such as but not limited to, a circular opening, a square or rectangular opening, a U-shaped opening, a narrow opening that only allows the penetrating member 14 to pass, an opening with more clearance on the sides, a slit, and the like.

The use of the sterility barrier 20 can facilitate the manufacture of disposable device 22. For example, a single sterility barrier 20 can be adhered, attached, or otherwise coupled to the disposable device 22 to seal many of the cavities 26 at one time. A sheet of analyte detecting members 16 can also be adhered, attached, or otherwise coupled to the disposable device 22 to provide many analyte detecting members 16 on or in the disposable device 22 at one time. During manufacturing of one embodiment of the present invention, the disposable device 22 can be loaded with penetrating members 14, sealed with sterility barrier 20 and a temporary layer (not shown) on the bottom where scaffolding 24 would later go, to provide a sealed environment for the penetrating members 14. This assembly with the temporary bottom layer is then taken to be sterilized. After sterilization, the assembly is taken to a clean room (or it can already be in a clear room or equivalent environment) where the temporary bottom layer is removed and the scaffolding 24 with analyte detecting members 16 is coupled to the disposable device 22. This process allows for the sterile assembly of the disposable device 22 with the penetrating members 14 using processes and/or temperatures that can degrade the accuracy or functionality of the analyte detecting members 16 on the scaffolding 24.

In some embodiments, more than one sterility barrier 20 can be used to seal the cavities 26. As examples of some embodiments, multiple layers can be placed over each cavity 26, half or some selected portion of the cavities 26 can be sealed with one layer with the other half or selected portion of the cavities sealed with another sheet or layer, different shaped cavities 26 can use different seal layer, or the like. The sterility barrier 20 can have different physical properties, such as those covering the penetrating members 14 near the end of the disposable device 22 can have a different color such as red to indicate to the user (if visually inspectable) that the user is down to say 10, 5, or other number of penetrating members before the cartridge should be changed out.

After actuation, the penetrating member 14 is returned into the disposable device 22 and is held therein in a manner so that it is not able to be used again. By way of example and not limitation, a used penetrating member 14 may be returned into the disposable member 22 and held by a launcher in position until the next lancing event. At the time of the next lancing, the launcher may disengage the used penetrating member with the disposable device 22 turned or indexed to the next clean penetrating member 14 such that the cavity 26 holding the used penetrating member is positioned so that it is not accessible to the user (i.e. turn away from a penetrating member exit opening). In some embodiments, the tip of a used penetrating member 14 may be driven into a protective stop that hold the penetrating member in place after use. The disposable device 22 is replaceable with a new disposable device 22 once all the penetrating members 14 have been used or at such other time or condition as deemed desirable by the user.

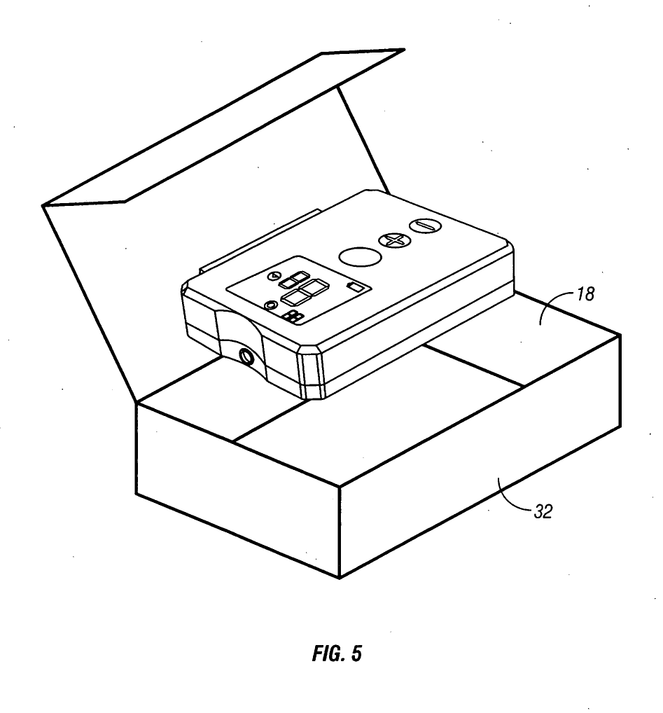

As shown in FIG. 4, a cassette 27 can be provided for housing the disposable device 22 and is sized to fit within the instrument housing 12.

The disposable device 22 can provide sterile environments for penetrating members 14 via the sterility barrier 20, seals, foils, covers, polymeric, or similar materials used to seal the cavities 26 and provide enclosed areas for the penetrating members 14 to rest in. In one embodiment, sterility barrier 20 is applied to one surface of the disposable device 20. Each cavity 26 may be individually sealed in a manner such that the opening of one cavity 26 does not interfere with the sterility in an adjacent or other cavity 26. Additionally, the disposable device 22 can include a moisture barrier 29.

The plurality of penetrating members 14 can be at least partially contained in the cavities 26 of the disposable device 22. The penetrating members 14 are slidably movable to extend outward from the disposable device 22 to penetrate tissue. The cavities 26 can each have a longitudinal opening that provides access to an elongate portion of the penetrating member 14. The sterility barrier 20 can cover the longitudinal openings. The sterility barrier 20 can be configured to be moved so that the elongate portion can be accessed by a gripper without touching the sterility barrier 20.

Referring again to FIG. 1, another aspect of the present invention will now be described. At least one gasket 28 on the instrument housing 12 can be provided to create a sealed air-tight environment inside the instrument housing 12 to create a seal. In various embodiments, the seal is formed around, each of analyte detecting member 16, the disposable device 22, around the instrument housing 12, and the like. The seal is broken only during lancing and blood sampling. A lid 30 can cover a penetrating member exit port. A block of desiccant 18 can be incorporated into the disposable device 22, and this desiccant 18 dries the air inside of the device 10. Individual analyte detecting members 16 in the disposable device 22 are not sealed from the environment in this embodiment. However, since these analyte detecting members 16 are inside of the device 10, and the air inside the device 10 is kept dry, the analyte detecting members 16 are still protected from humidity.

Once a new disposable device 22 is inserted, the entire inside of the device 10 is sealed from the outside environment. The disposable device 22 can be packaged to come with a large block or other sufficient size of desiccant 18 to desiccate the entire interior volume of the device 10. The desiccant 18 can assume a variety of forms including but not limited to a disc of desiccant 18 that can be placed under the disposable device 22. In other embodiments, the disposable device 22 can be part of the cassette 27 that can house the desiccant 18 and the cassette 27 can have a block of desiccant 18 in the cassette 27. By way of example and not limitation, the desiccant can be molded to the wall of the cassette or can simply be housed in the cassette 27. These applications will work because the interior of the instrument will be sealed from the outside environment when the device is not in use or configured in a mode that is ready for use.

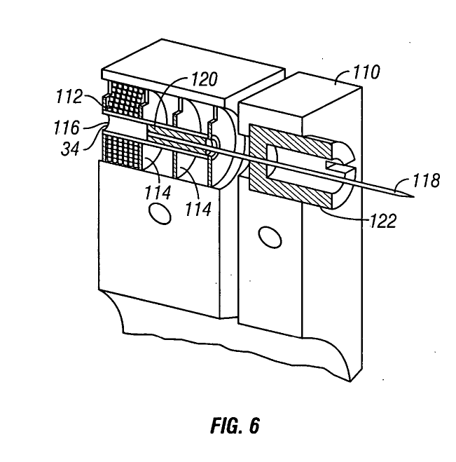

FIG. 5 shows an embodiment where the device 10 is unsealed, with unsealed analyte detecting members 16, but a case 32 is provided. The case 32 can be lined with or otherwise designed to contain the desiccant 18. Except during the brief periods when the user is positioning the device 10 for a lancing event and glucose measurement, the device 10 is stored in the case 32. The instrument (and/or the case) can be designed to determine if it is in the case 32 and send warnings or reminders to the user to place the instrument into the proper storage condition. The alarm can also be used to remind the user to close various doors or caps.

In one embodiment, the desiccant 18 can be designed to keep the analyte detecting members sufficiently dry for 90 days in a normal climate condition. Additionally, since every time the device is used is that a drop of blood is left inside the desiccated environment (on the analyte detecting member). An amount of desiccant sufficient to reduce the spike in humidity after each test is desired. In one embodiment, about 5 cc of desiccant is used. Other embodiments can use greater volumes to more quickly absorb the spike in humidity the occurs after blood is introduced into the desiccated environment.

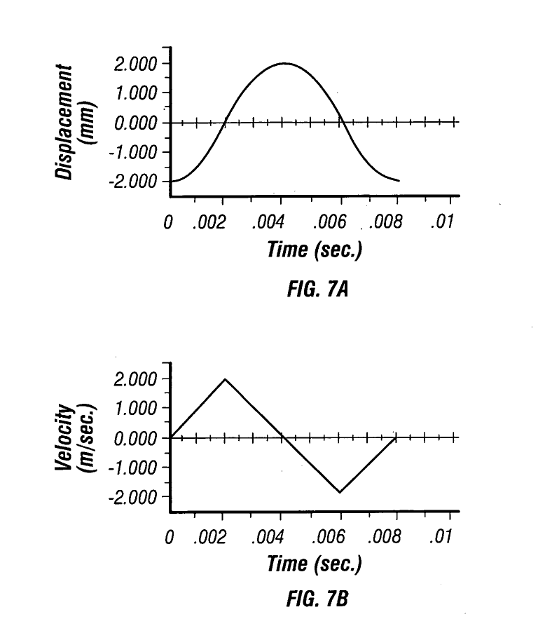

In one embodiment of the present invention, a device, generally denoted as 34, is included to provide controlled velocity and depth of penetration of the penetrating members 14, as shown in Figure. Device 34 can be any variety of different penetrating member drivers. It is contemplated that the device 34 can be spring based, solenoid based, magnetic driver based, nanomuscle based, or based on any other mechanism useful in moving a penetrating member along a path into tissue. It should be noted that the present invention is not limited by the type of driver used with a penetrating member feed mechanism. One suitable penetrating member driver for use with the present invention is shown in FIG. 6. This is an embodiment of a solenoid type electromagnetic driver that is capable of driving an iron core or slug mounted to the penetrating member assembly using a direct current (DC) power supply. The electromagnetic driver includes a driver coil pack that is divided into three separate coils along the path of the penetrating member, two end coils and a middle coil. Direct current is alternated to the coils to advance and retract the penetrating member. Although the driver coil pack is shown with three coils, any suitable number of coils can be used, for example, 4, 5, 6, 7 or more coils can be used.

Referring to the embodiment of FIG. 6, the stationary iron housing 110 can contain the driver coil pack with a first coil 112 flanked by iron spacers 114 which concentrate the magnetic flux at the inner diameter creating magnetic poles. The inner insulating housing 116 isolates the penetrating member 18 and iron core 120 from the coils and provides a smooth, low friction guide surface. The penetrating member guide 122 further centers the penetrating member 118 and iron core 120. The penetrating member 118 is protracted and retracted by alternating the current between the first coil 12, the middle coil, and the third coil to attract the iron core 120. Reversing the coil sequence and attracting the core and penetrating member back into the housing retracts the penetrating member. The penetrating member guide 122 also serves as a stop for the iron core 120 mounted to the penetrating member 118.

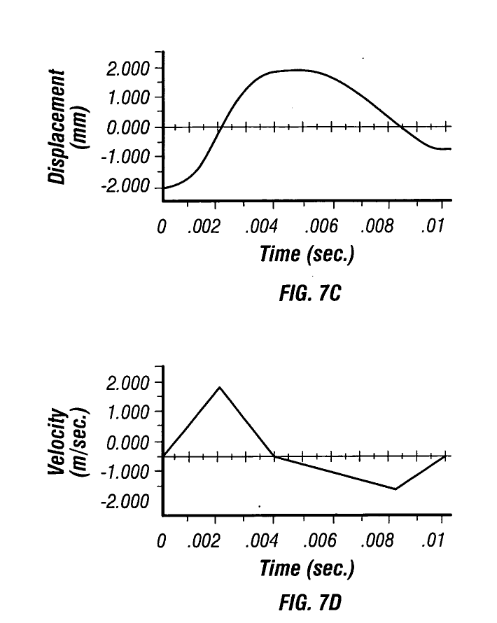

As discussed above, tissue penetration devices 14 which employ spring or cam driving methods have a symmetrical or nearly symmetrical actuation displacement and velocity profiles on the advancement and retraction of the penetrating member as shown in FIGS. 7(a) through 7(d). In most of the available lancet devices, once the launch is initiated, the stored energy determines the velocity profile until the energy is dissipated. Controlling impact, retraction velocity, and dwell time of the penetrating member within the tissue can be useful in order to achieve a high success rate while accommodating variations in skin properties and minimize pain. Advantages can be achieved by taking into account of the fact that tissue dwell time is related to the amount of skin deformation as the penetrating member tries to puncture the surface of the skin and variance in skin deformation from patient to patient based on skin hydration.

In this embodiment, the ability to control velocity and depth of penetration can be achieved by use of a controllable force driver where feedback is an integral part of driver control. Such drivers can control either metal or polymeric penetrating members or any other type of tissue penetration element. The dynamic control of such a driver is illustrated in FIG. 7(c) which illustrates an embodiment of a controlled displacement profile and FIG. 7(d) which illustrates an embodiment of a the controlled velocity profile. These are compared to Figures (a) and (b), which illustrate embodiments of displacement and velocity profiles, respectively, of a harmonic spring/mass powered driver. Reduced pain can be achieved by using impact velocities of greater than about 2 m/s entry of a tissue penetrating element, such as a lancet, into tissue. Other suitable embodiments of the penetrating member driver are described in commonly assigned, copending U.S. patent application Ser. No. 10/127,395, (Attorney Docket No. 38187-2551) filed Apr. 19, 2002 and previously incorporated herein.

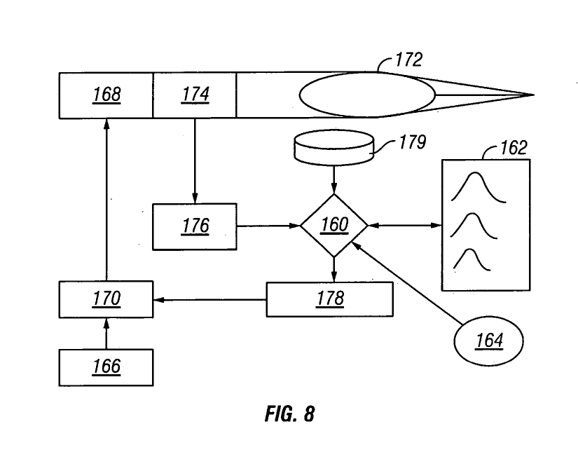

FIG. 8 illustrates the operation of a feedback loop using a processor 160. The processor 160 stores profiles 162 in non-volatile memory. A user inputs information 164 about the desired circumstances or parameters for a lancing event. The processor 160 selects a driver profile 162 from a set of alternative driver profiles that have been preprogrammed in the processor 160 based on typical or desired tissue penetration device performance determined through testing at the factory or as programmed in by the operator. The processor 160 can customize by either scaling or modifying the profile based on additional user input information 164. Once the processor has chosen and customized the profile, the processor 160 is ready to modulate the power from the power supply 66 to the penetrating member driver 168 through an amplifier 170. The processor 60 can measure the location of the penetrating member 172 using a position sensing mechanism 174 through an analog to digital converter 176 linear encoder or other such transducer. Examples of position sensing mechanisms have been described in the embodiments above and can be found in the specification for commonly assigned, copending U.S. patent application Ser. No. 10/127,395, (Attorney Docket No. 38187-2551) filed Apr. 19, 2002 and previously incorporated herein. The processor 160 calculates the movement of the penetrating member by comparing the actual profile of the penetrating member to the predetermined profile. The processor 160 modulates the power to the penetrating member driver 168 through a signal generator 178, which can control the amplifier 170 so that the actual velocity profile of the penetrating member 14 does not exceed the predetermined profile by more than a preset error limit. The error limit is the accuracy in the control of the penetrating member 14.

After the lancing event, the processor 160 can allow the user to rank the results of the lancing event. The processor 160 stores these results and constructs a database 180 for the individual user. Using the database 179, the processor 160 calculates the profile traits such as degree of painlessness, success rate, and blood volume for various profiles 162 depending on user input information 164 to optimize the profile to the individual user for subsequent lancing cycles. These profile traits depend on the characteristic phases of penetrating member advancement and retraction. The processor 160 uses these calculations to optimize profiles 162 for each user. In addition to user input information 64, an internal clock allows storage in the database 179 of information such as the time of day to generate a time stamp for the lancing event and the time between lancing events to anticipate the user's diurnal needs. The database 179 stores information and statistics for each user and each profile that particular user uses.

In addition to varying the profiles, the processor 160 can be used to calculate the appropriate penetrating member diameter and geometry suitable to realize the blood volume required by the user. For example, if the user requires about 1-5 microliter volume of blood, the processor 160 can select a 200 micron diameter penetrating member to achieve these results. For each class of lancet, both diameter and lancet tip geometry, is stored in the processor 160 to correspond with upper and lower limits of attainable blood volume based on the predetermined displacement and velocity profiles.

The lancing device is capable of prompting the user for information at the beginning and the end of the lancing event to more adequately suit the user. The goal is to either change to a different profile or modify an existing profile. Once the profile is set, the force driving the penetrating member is varied during advancement and retraction to follow the profile. The method of lancing using the lancing device comprises selecting a profile, lancing according to the selected profile, determining lancing profile traits for each characteristic phase of the lancing cycle, and optimizing profile traits for subsequent lancing events.

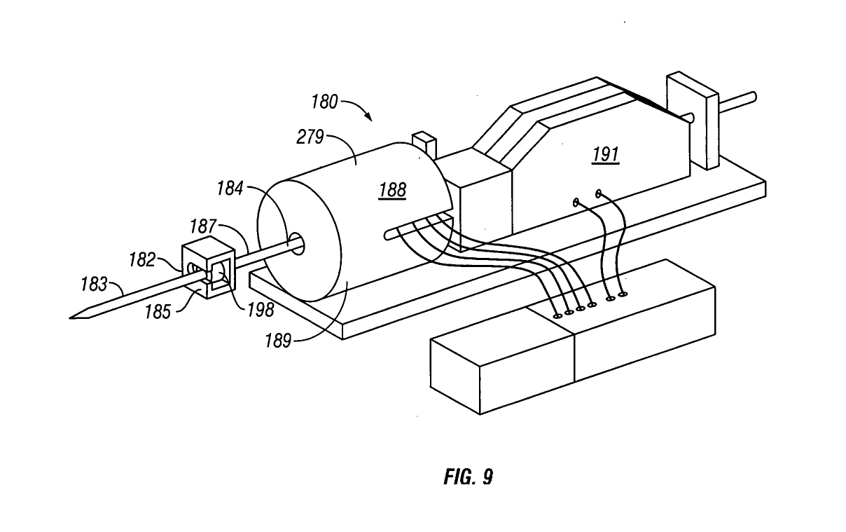

FIG. 9 illustrates an embodiment of a tissue penetration device, more specifically, a lancing device 180 that includes a controllable driver 279 coupled to a tissue penetration element 14. The lancing device 180 has a proximal end 181 and a distal end 182. At the distal end 182 is the tissue penetration element in the form of a penetrating member 183, which is coupled to an elongate coupler shaft 184 by a drive coupler 185. The elongate coupler shaft 184 has a proximal end 186 and a distal end 187. A driver coil pack 188 is disposed about the elongate coupler shaft 184 proximal of the penetrating member 183. A position sensor 191 is disposed about a proximal portion 192 of the elongate coupler shaft 184 and an electrical conductor 194 electrically couples a processor 193 to the position sensor 191. The elongate coupler shaft 184 driven by the driver coil pack 188 controlled by the position sensor 191 and processor 193 form the controllable driver, specifically, a controllable electromagnetic driver.

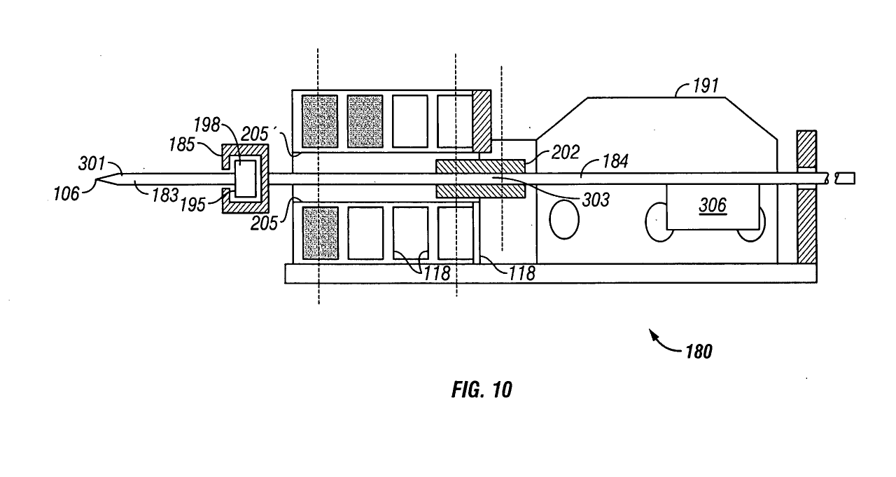

Referring to FIG. 10, the lancing device 180 can be seen in more detail, in partial longitudinal section. The penetrating member 183 has a proximal end 195 and a distal end 196 with a sharpened point at the distal end 196 of the penetrating member 183 and a drive head 198 disposed at the proximal end 195 of the penetrating member 183. A penetrating member shaft 301 is disposed between the drive head 198 and the sharpened point 197. The penetrating member shaft 301 can be comprised of stainless steel, or any other suitable material or alloy and have a transverse dimension of about 0.1 to about 0.4 mm. The penetrating member shaft can have a length of about 3 mm to about 50 mm, specifically, about 15 mm to about 20 mm. The drive head 198 of the penetrating member 183 is an enlarged portion having a transverse dimension greater than a transverse dimension of the penetrating member shaft 301 distal of the drive head 198. This configuration allows the drive head 198 to be mechanically captured by the drive coupler 185. The drive head 198 can have a transverse dimension of about 0.5 to about 2 mm.

A magnetic member 202 is secured to the elongate coupler shaft 184 proximal of the drive coupler 185 on a distal portion of the elongate coupler shaft 184. The magnetic member 202 is a substantially cylindrical piece of magnetic material having an axial lumen 304 extending the length of the magnetic member 202. The magnetic member 202 has an outer transverse dimension that allows the magnetic member 202 to slide easily within an axial lumen 205 of a low friction, possibly lubricious, polymer guide tube 205′ disposed within the driver coil pack 188. The magnetic member 202 can have an outer transverse dimension of about 1.0 to about 5.0 mm, specifically, about 2.3 to about 2.5 mm. The magnetic member 202 can have a length of about 3.0 to about 5.0 mm, specifically, about 4.7 to about 4.9 mm. The magnetic member 202 can be made from a variety of magnetic materials including ferrous metals such as ferrous steel, iron, ferrite, or the like. The magnetic member 202 can be secured to the distal portion 303 of the elongate coupler shaft 184 by a variety of methods including adhesive or epoxy bonding, welding, crimping or any other suitable method.

Proximal of the magnetic member 202, an optical encoder flag 306 is secured to the elongate coupler shaft 184. The optical encoder flag 306 is configured to move within a slot in the position sensor 191. The slot can have separation width of about 1.5 to about 2.0 mm. The optical encoder flag 306 can have a length of about 14 to about 18 mm, a width of about 3 to about 5 mm and a thickness of about 0.04 to about 0.06 mm.

The optical encoder flag 306 interacts with various optical beams generated by LEDs disposed on or in the position sensor body portions in a predetermined manner. The interaction of the optical beams generated by the LEDs of the position sensor 191 generates a signal that indicates the longitudinal position of the optical flag 306 relative to the position sensor 191 with a substantially high degree of resolution. The resolution of the position sensor 191 can be about 200 to about 400 cycles per inch, specifically, about 350 to about 370 cycles per inch. The position sensor 191 can have a speed response time (position/time resolution) of 0 to about 120,000 Hz, where one dark and light stripe of the flag constitutes one Hertz, or cycle per second. The position of the optical encoder flag 306 relative to the magnetic member 202, driver coil pack 188 and position sensor 191 is such that the optical encoder 191 can provide precise positional information about the penetrating member 183 over the entire length of the penetrating member's power stroke.

An optical encoder that is suitable for the position sensor 191 is a linear optical incremental encoder, model HEDS 9200, manufactured by Agilent Technologies. The model HEDS 9200 can have a length of about 20 to about 30 mm, a width of about 8 to about 12 mm, and a height of about 9 to about 11 mm. Although the position sensor 191 illustrated is a linear optical incremental encoder, other suitable position sensor embodiments could be used, provided they posses the requisite positional resolution and time response. The HEDS 9200 is a two channel device where the channels are 90 degrees out of phase with each other. This results in a resolution of four times the basic cycle of the flag. These quadrature outputs make it possible for the processor to determine the direction of penetrating member travel. Other suitable position sensors include capacitive encoders, analog reflective sensors, such as the reflective position sensor discussed above, and the like.

While the invention has been described and illustrated with reference to certain particular embodiments thereof, those skilled in the art will appreciate that various adaptations, changes, modifications, substitutions, deletions, or additions of procedures and protocols can be made without departing from the spirit and scope of the invention. For example, with any of the above embodiments, the shield or other punch can be adapted for use with other cartridges disclosed herein or in related applications. With any of the above embodiments, the methods for storage can be used with analyte sampling devices, analyte sampling and measurement devices, and/or analyte measurement devices. The use is not restricted. With any of the above embodiments, the lids can be flip up or slide. They can be motorized or user actuated. With any of the above embodiments, the gasket can also be designed for compression. The sliding lids are designed to compress the O-ring to provide a seal.

The publications discussed or cited herein are provided solely for their disclosure prior to the filing date of the present application. Nothing herein is to be construed as an admission that the present invention is not entitled to antedate such publication by virtue of prior invention. Further, the dates of publication provided can be different from the actual publication dates which can need to be independently confirmed. All publications mentioned herein are incorporated herein by reference to disclose and describe the structures and/or methods in connection with which the publications are cited.

Expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A fluid sampling device comprising:
an instrument housing;
a plurality of penetrating members in the instrument housing;
a plurality of analyte detecting members, each of an analyte detecting member coupled to a penetrating member; and
a desiccant material inside the instrument housing and positioned external to the plurality of penetrating members, the desiccant material reducing humidity.

2. The device of claim 1, further comprising:
a sterility barrier configured to provide sterile environments for the plurality of penetrating members.

3. The device of claim 1, wherein the desiccant is present in an amount of no more than 50 mm³ per each of an analyte detecting member.

4. The device of claim 1, wherein the desiccant is present in an amount of 10-20 mm³ per each of an analyte detecting member.

5. The device of claim 1, wherein the desiccant is present in an amount of 10-15 mm³ per each of an analyte detecting member.

6. The device of claim 1, wherein the desiccant is present in an amount of at least 1 mm³ per each of an analyte detecting member.

7. The device of claim 1, wherein the desiccant is selected from at least one of a molecular sieve, a silica gel or a clay.

8. The device of claim 7, wherein the molecular sieve is mixed with a polymeric binder.

9. The device of claim 1, further comprising
a scaffolding that supports the plurality of analyte detecting members.

10. The device of claim 9, wherein the scaffolding holds the desiccant.

11. The device of claim 10, wherein the scaffolding includes a desiccant for each of an analyte detecting member.

12. The device of claim 1, wherein the desiccant is present as a desiccant block inside of the instrument housing.

13. The device of claim 10, wherein the desiccant is molded and inserted into the scaffolding.

14. The device of claim 1, wherein the desiccant is coupled with the scaffolding.

15. The device of claim 1, wherein the desiccant and the scaffolding are co-molded simultaneously.

16. The device of claim 1, wherein the scaffolding and the desiccant are co-molded sequentially.

17. The device of claim 1, wherein the plurality of analyte detecting members and the plurality of penetrating members form a disposable device.

18. The device of claim 17, wherein the desiccant material is configured to be replaced when the disposable device is replaced from the instrument housing.

19. The device of claim 14, wherein the desiccant material is external to the analyte detecting members.

20. The device of claim 14, wherein the desiccant is on at least a portion of the analyte detecting members.

21. The device of claim 17, wherein the disposable device includes a plurality of cavities.

22. The device of claim 17, further comprising:
a cassette for housing the disposable device and sized to fit within the instrument housing.

23. The device of claim 22, wherein the plurality of penetrating members are at least partially contained in the cavities of the disposable device, wherein the penetrating members are slidably movable to extend outward from the disposable device to penetrate tissue, the cavities each having a longitudinal opening providing access to an elongate portion of the penetrating member.

24. The device of claim 23, wherein a sterility barrier covers a plurality of the longitudinal openings, wherein the sterility barrier is configured to be moved so that the elongate portion can be accessed by a gripper without touching the sterility barrier.

25. The device of claim 24, further comprising:
at least one gasket on the instrument housing to create a sealed air-tight environment inside the instrument housing.

26. The device of claim 14, wherein each of an analyte detecting members are stored in an air tight desiccated environment.

27. The device of claim 14, wherein an air seal is formed around each of an analyte detecting member.

28. The device of claim 17, wherein an air tight seal is formed around the disposable device.

29. The device of claim 1, wherein an air tight seal is formed around the instrument housing.

30. The device of claim 1, wherein the instrument housing is in a sealed case.

31. The device of claim 1 further comprising:
a case sized to contain the instrument housing, the case containing the desiccant and providing a sealed environment when closed.

32. The device of claim 1, further comprising:
a device that provides controlled velocity and depth of penetration of the penetrating members.

33. A device for use in penetrating tissue to obtain a body fluid sample, comprising:
a instrument housing;
a plurality of penetrating members;
a plurality of analyte detecting members, each of an analyte detecting member being associated with a penetrating member; and
a case sized to contain the instrument housing; and
a desiccant material inside the instrument housing or the case, the desiccant material being positioned external to the plurality of penetrating members.

34. The device of claim 33, wherein the desiccant is present in an amount of no more than 50 mm³ per each of an analyte detecting member.

35. The device of claim 33, wherein the desiccant is present in an amount of 10-20 mm³ per each of an analyte detecting member.

36. The device of claim 33, wherein the desiccant is present in an amount of 10-15 mm³ per each of an analyte detecting member.

37. The device of claim 33, wherein the desiccant is present in an amount of at least 1 mm³ per each of an analyte detecting member.

38. The device of claim 33, wherein the desiccant is selected from at least one of a molecular sieve, a silica gel or a clay.

39. The device of claim 38, wherein the molecular sieve is mixed with a polymeric binder.

40. The device of claim 33, further comprising
a scaffolding that supports the plurality of analyte detecting members.

41. The device of claim 40, wherein the scaffolding holds the desiccant.

42. The device of claim 41, wherein the scaffolding includes a desiccant for each of an analyte detecting member.

43. The device of claim 1, wherein the desiccant is present as a desiccant block inside of the instrument housing.

44. The device of claim 41, wherein the desiccant is molded and inserted into the scaffolding.

45. The device of claim 33, wherein the desiccant is coupled with the scaffolding.

46. The device of claim 33, wherein the desiccant and the scaffolding are co-molded simultaneously.

47. The device of claim 33, wherein the scaffolding and the desiccant are co-molded sequentially.

48. The device of claim 33, wherein the plurality of analyte detecting members and the plurality of penetrating members form a disposable device.

49. The device of claim 48, wherein the desiccant material is configured to be replaced when the disposable device is replaced from the instrument housing.

50. The device of claim 43, wherein the desiccant material is external to the analyte detecting members.

51. The device of claim 43, wherein the desiccant is on at least a portion of the analyte detecting members.

52. The device of claim 48, wherein the disposable device includes a plurality of cavities.

53. The device of claim 48, further comprising:
a cassette for housing the disposable device and sized to fit within the instrument housing.

54. The device of claim 53, wherein the plurality of penetrating members are at least partially contained in the cavities of the disposable device, wherein the penetrating members are slidably movable to extend outward from the disposable device to penetrate tissue, the cavities each having a longitudinal opening providing access to an elongate portion of the penetrating member.

55. The device of claim 54, wherein a sterility barrier covers a plurality of the longitudinal openings, wherein the sterility barrier is configured to be moved so that the elongate portion can be accessed by a gripper without touching the sterility barrier.

56. The device of claim 55, further comprising:
at least one gasket on the instrument housing to create a sealed air-tight environment inside the instrument housing.

57. The device of claim 43, wherein each of an analyte detecting members are stored in an air tight desiccated environment.

58. The device of claim 43, wherein an air seal is formed around each of an analyte detecting member.

59. The device of claim 48, wherein an air tight seal is formed around the disposable device.

60. The device of claim 33, wherein an air tight seal is formed around the instrument housing.

61. The device of claim 33, wherein the instrument housing is in a sealed case.

62. The device of claim 33, further comprising:
a device that provides controlled velocity and depth of penetration of the penetrating members.

63. A method to determine an amount on an analyte in a body fluid sample by a user, comprising:
(a) providing an analyte measuring device that has a instrument housing, a plurality of penetrating members in the instrument housing, a plurality of analyte detecting members, and a desiccant material inside the instrument housing and positioned external to the plurality of penetrating members;
(b) desiccating the plurality of analyte detecting members with the desiccant that is external to the plurality of penetrating members;
(c) presenting a penetrating member and unused analyte detecting member of the analyte measurement device into an active position;
(d) firing the penetrating member to prick the skin and bring a fluid sample to the analyte detecting member; and
(e) measuring the analyte level.

64. The method of claim 63, wherein steps (a) through (e) are performed without the user directly handling the penetrating member to obtain a fresh penetrating member or load the penetrating member

65. The method of claim 63, wherein steps (a) through (e) are performed without the user coding the analyte measurement device.

66. The method of claim 63, wherein blood is applied to an analyte detection member during lancing.

67. The method of claim 66, wherein the application of blood to an analyte detection member during lancing occurs without removal and disposal of penetrating members from the analyte measurement device.

68. The method of claim 63, wherein steps (a) through (e) are performed without a separate step of apply blood to a analyte detection member after lancing.

69. The method of claim 63, wherein step (d) is performed without milking a wound.

70. The method of claim 63, wherein step (d) is performed using at least one of a penetrating member driver selected from, spring based, electro-mechanical based, magnetic driver based, and nanomuscle based.

71. The method of claim 63, wherein step (d) is performed with controlled velocity and depth of penetration.

72. The method of claim 63, further comprising:
returning the analyte measuring device to a storage condition without having to dispose of a used penetrating member or used analyte detecting members.

73. The method of claim 63, wherein the analyte measuring device is ready for the next lancing event without having to dispose of the used penetrating member or the used analyte detecting member.
